(19) United States
(12) Patent Application Publication
(10) Pub. No.: US 2002/0164732 A1
(43) Pub. Date: Nov. 7, 2002

(54) RESISTANCE MECHANISMS TO IC TYPE R/M BACTERIOPHAGES OF LACTIC ACID BACTERIA

(76) Inventors: MARIE-CHRISTINE CHOPIN, PARIS (FR); FLORENCE CLIER, JOUY-EN-JOSAS (FR); S, DUSKO EHRLICH, PARIS (FR); MICHEL GAUTIER, RENNES (FR); CATHERINE SCHOULER, CHARENTILLY (FR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 09/486,734

(22) PCT Filed: Sep. 1, 1998
(86) PCT No.: PCT/FR98/01873

(30) Foreign Application Priority Data
Sep. 2, 1997 (FR) 97/10885

(51) Int. Cl. C12Q 1/68

(52) U.S. Cl. 435/183; 435/193; 435/243; 435/6; 536/23.2; 530/350

(57) ABSTRACT

The invention concerns polypeptides constituting subunits of a resistance mechanism to Ic type R/M bacteriophages, active against the phages of lactic acid bacteria, and nucleic acid sequences coding for said polypeptides. The expression of said polypeptides in lactic acid bacteria enables to increase resistance to the attacks of bacteriophages.

The invention relates to systems of resistance to type Ic R/M bacteriophages, which are active on the bacteriophages of lactic acid bacteria.

Producing lactic acid bacteria resistant to attack by bacteriophages is of great value in the field of industrial fermentation.

With this aim, it is generally proposed:

either to select mutants, natural or obtained by mutagenesis, which are bacteriophage-resistant;

or to use vectors carrying genes encoding mechanisms of phage-resistance, to transfer these genes into initially non-resistant strains.

The mechanisms of phage resistance are grouped into 3 principal classes according to the step of the bacteriophage reproductive cycle with which they interfere:

the mechanisms grouped under the name “mechanisms of interference with adsorption ”, delay the adsorption of the phage onto the bacterium;

the mechanisms named “abortive infection mechanisms” (Abi) block phage multiplication and bring about the death of the infected bacterium before this bacterium produces viral particles capable of infecting other cells;

the mechanisms named “restriction/modification (R/M) mechanisms ”, degrade the phage DNA as soon as it enters the bacterium. These mechanisms combine a restriction endonuclease and a methylase whose function is to protect the bacterial genome by modifying the sequences of this genome which might constitute target sequences for the endonuclease.

3 principal types of R/M mechanism (types I, II and III) have been described [for review, cf. for example Bickle and Kruger, Microbiological Reviews, 57, pp. 434-450 (1993)]. The characteristics of type I and II R/M mechanisms are briefly recalled below:

The type II R/M mechanisms are the most common; they consist of two distinct enzymes, a methylase and an endonuclease, which are independently active and which recognize a common target-sequence.

The type I R/M mechanisms were discovered principally in enterobacteria, then were also demonstrated in a few Gram⁺ bacteria (Bacillus subtilis and Mycobacterium pulmonis) and in archaebacteria.

In these mechanisms, the methylase (M, or HsdM subunit) and the endonuclease (R or HsdR subunit) each form one subunit of a multifunctional enzyme complex in which they are associated with a third protein (S, or HsdS subunit), which is responsible for the recognition of specific sequences by the enzyme complex.

These three proteins are the products of genes named hsdM, hsdR and hsdS, respectively. Genetic complementation experiments and the comparison of the sequences of these genes have allowed the type I R/M mechanisms to be divided into 4 separate families having only very weak homology between them: Ia, Ib, Ic and Id.

Moreover, it has been observed that the type I R/M mechanisms could acquire a new specificity following recombination events in the hsdS gene [FULLER-PACE and MURRAY, Proc. Natl. Acad. Sci. USA, 83, pp. 9368-9372, (1986); SHARP et al., Proc. Natl. Acad. Sci. USA, 89, pp. 9836-9840, (1992); GUBLER et al. EMBO J., 11, pp. 233-240, (1992)].

The R/M mechanisms which have so far been characterized in the lactic acid bacteria and whose use has been proposed for rendering these bacteria more resistant to bacteriophages, are type II R/M mechanisms [FITZGERALD et al., Nucleic Acids Research, 10, pp. 8171-8179, (1982); NYENGAARD et al., Gene, 136, pp. 371-372, (1993); TOWNEY et al., Gene, 136, pp. 205-209, (1993); DAVIS et al., Appl. Environ. Microbiol., 59, pp. 777-785, (1995); NYENGAARD et al., Gene, 157, pp. 13-18, (1995); O'SULLIVAN et al., J. Bacteriol., 177, pp. 134-143 (1995); MOINEAU et al., Appl. Environ. Microbiol., 61, pp. 2193-2202, (1995)].

One of the principal disadvantages of using this type of mechanism is the appearance of resistant phages, due in particular to the fact that the methylase of the R/M system recognizes and modifies a certain proportion of the phage DNA molecules, which may then escape the action of the endonuclease. This phenomenon appears in particular during sustained use under industrial fermentation conditions. To limit this phenomenon, the combination of several R/M mechanisms recognizing various target sequences has been proposed [JOSEPHSEN and KLAENHAMMER, Plasmid, 23, 71-75 (1989)]; it is, therefore, particularly desirable to isolate novel mechanisms, of different specificity to that of the mechanisms already known.

The inventors have now isolated novel type Ic R/M mechanisms, which are active in lactic acid bacteria, and have expressed the genes encoding the different constituents thereof.

A subject of the present invention is a polypeptide which constitutes one of the HsdR, HsdM, or HsdS subunits of a mechanism of resistance to type Ic R/M bacteriophages, characterized in that said mechanism is active against the phages of lactic acid bacteria.

According to a preferred embodiment of a polypeptide which constitutes an HsdR subunit in accordance with the present invention, it comprises the following sequence (I) (represented in 1-letter code):

KRLARK

According to a preferred embodiment of a polypeptide which constitutes an HsdM subunit in accordance with the present invention, it comprises at least one of the following sequences (represented in a 1-letter code):

the sequence (II):

GLX₁FYKYLS

in which X₁ represents I or L,

the sequence (III):

ENDX₂NLNIPRYVDTFEEEE

in which X₂ represents Y or F

According to a preferred embodiment of a polypeptide which constitutes an HsdS subunit in accordance with the present invention, it comprises:

an N-terminal domain of about 150 to 180 amino acids, comprising the following sequence (IV):

PX₃LRFX₄GFTX₅DDWEERKX₆

in which X₃ represents E or Q, X₄ represents E, P, D or K, X₅ represents N or D, X₆ represents L or F;

a central domain of about 50 to 80 amino acids, comprising the following sequence (V):

EQX₇KIGX₈FFKX₉LDX₁₀TIX₁₁LHQRKLDLLKEQKKGYX₁₂QKMFPKNGX₁₃KX₁₄PELRFAX₁₅FADDWEX₁₆RKLG

in which X₇ represents R or Q, X₈ represents S, N or L, X₉ represents E, H or Q, X₁₀ represents A, D or N, X₁₁ represents A or V, X₁₂ represents L or F, X₁₃ represents A or S, X₁₄ represents I or V, X₁₅ represents E or G, X₁₆ represents D or E;

a C-terminal domain of about 160 to 200 amino acids, comprising the following sequence (VI):

EQX₁₅X₁₆IGSFFKQLDX₁₇TIX₁₈LHQRKLX₁₉

in which X₁₅ represents K or Q, X₁₆ represents K or Q, X₁₇ represents N or D, X₁₈ represents T, A or V, X₁₉ represents A or D;

and/or the following sequence (VII):

GFLQKMFX₂₀

in which X₂₀ represents V or H.

The present invention encompasses in particular:

the HsdR polypeptides corresponding to one of the sequences, respectively, represented in the annexed sequence listing under the numbers SEQ ID NO: 2 and SEQ ID NO: 4;

the HsdM polypeptides corresponding to one of the sequences, respectively, represented in the annexed sequence listing under the numbers SEQ ID NO: 6 and SEQ ID NO: 8;

the HsdS polypeptides corresponding to one of the sequences, respectively, represented in the annexed sequence listing under the numbers SEQ ID NO: 10, SEQ ID NO: 12, SEQ ID NO: 14, SEQ ID NO: 16.

Association of any one of the HsdR polypeptides with any one of the HsdM polypeptides and any one of the HsdS polypeptides in accordance with the invention produces an enzyme complex constituting a type Ic R/M mechanism which is active in lactic acid bacteria. The specificity of action of this mechanism is conditioned by the HsdS polypeptide. The sequences (IV), (V), (VI) and (VII) represent conserved regions of the HsdS polypeptides in lactic acid bacteria (and in particular in lactococci), which are probably involved in the association of these peptides with the HsdR and HsdM polypeptides; these conserved regions are separated by variable regions which are involved in the recognition of specific nucleotide sequences.

A subject of the present invention is also the nucleic acid sequences encoding the polypeptides defined above, and complementary sequences thereof.

DNA sequences in accordance with the invention are for example represented by:

the hsdR sequences SEQ ID NO: 1 and SEQ ID NO: 3, which code respectively for the polypeptides SEQ ID NO: 2 and SEQ ID NO: 4;

the hsdM sequences SEQ ID NO: 5 and SEQ ID NO: 7, which code respectively for the polypeptides SEQ ID NO: 6 and SEQ ID NO: 8;

the hsdS sequences SEQ ID NO: 9, SEQ ID NO: 11, SEQ ID NO: 13 and SEQ ID NO: 15, which code respectively for the polypeptides SEQ ID NO: 10, SEQ ID NO: 12, SEQ ID NO: 14 and SEQ ID NO: 16.

A subject of the present invention is also nucleic acid fragments of at least 18 bp which are homologous or complementary to all or part of a nucleic acid sequence encoding an HsdR, HsdM or HsdS polypeptide in accordance with the invention.

These fragments may in particular be used as hybridization probes and/or amplification primers, for detecting and selecting strains of lactic acid bacteria, or plasmids harboured by these strains, which contain at least one sequence encoding an HsdR, HsdM or HsdS polypeptide in accordance with the invention, and for isolating and/or cloning said sequence from a strain or a plasmid selected in this way.

Preferred subfragments are those which are situated within sequences encoding regions conserved between the HsdR subunits, between the HsdM subunits or between the HsdS subunits of the type Ic R/M mechanisms in accordance with the invention.

For example:

for selecting strains of lactic acid bacteria containing a sequence encoding an HsdR subunit, and/or for cloning said sequence, at least one oligonucleotide is advantageously used which is homologous or complementary to a sequence encoding at least 6 consecutive amino acids from one of the following peptides (represented in 1-letter code):

TGSGKT

KRLARK

LLTGFDS

which correspond to conserved regions of the HsdR subunits in L. lactis, identified by the inventors from the sequences SEQ ID NO: 2 and SEQ ID NO: 4

for selecting strains of lactic acid bacteria containing a sequence encoding an HsdM subunit, and/or for cloning said sequence, at least one oligonucleotide is advantageously used which is homologous or complementary to a sequence encoding at least 6 consecutive amino acids from one of the following peptides:

FYKYLS

LARMNL

LPHGVLFRGAAE

NLNIPRYVDTFEEEE

which correspond to conserved regions of the HsdM subunits in L lactis, identified by the Inventors by aligning the sequences SEQ ID NO: 6 and SEQ ID NO: 8;

for selecting strains of lactic acid bacteria containing a sequence encoding an HsdS subunit, and/or for isolating and cloning said sequence, at least one oligonucleotide is advantageously used which is homologous or complementary to a sequence encoding at least 6 consecutive amino acids from one of the following peptides:

DDWEERK

LHQRKLDLLKEQKKGY

QKMFPKNG

PELRFA

FADDWE

IGSFFKQLD

GFLQKMF

which correspond to conserved regions of the HsdS subunits in L. lactis, identified by the Inventors from the sequences SEQ ID NO: 10, SEQ ID NO: 12, SEQ ID NO: 14 and SEQ ID NO: 16.

The DNA sequences in accordance with the invention may be used to express, in a lactic acid bacterium, one or more type Ic R/M mechanisms, in order to increase its bacteriophage resistance.

In accordance with the invention, to allow the expression in a bacterium, in particular in a lactic acid bacterium, of at least one mechanism of resistance to type Ic R/M bacteriophages of lactic acid bacteria, transformation of said bacteria with at least one DNA sequence in accordance with the invention is carried out.

The process in accordance with the invention may be carried out in different ways. For example, if the host bacterium does not naturally contain a sequence which encodes one of the HsdR, HsdM or HsdS subunits, it has to be transformed with at least three DNA sequences, each of which encodes one of these polypeptides. If, on the other hand, the host bacterium already contains at least one sequence (carried by the bacterial chromosome or by a plasmid) encoding one of the HsdR, HsdM or HsdS subunits, it is sufficient to transform it with a sequence or sequences encoding the missing subunit(s).

Advantageously, from the same strain of host bacterium which contains at least one sequence encoding an HsdR subunit and at least one sequence encoding an HsdM subunit, it is possible, by transformation with different sequences encoding HsdS subunits of different specificity, to produce strains having different resistance properties, and to limit the emergence of phages which evade the mechanism of resistance.

Advantageously, to widen the spectrum of bacteriophage resistance, sequences encoding HsdS subunits of different specificity may be simultaneously used in the same host bacterium. With the same aim, a mechanism of bacteriophage resistance chosen from the Abi mechanisms and the type II R/M mechanisms, may also be expressed in the transformed bacterium. In this case, it is possible to use a host bacterium already comprising at least one sequence encoding an Abi mechanism and/or at least one sequence encoding a type II R/M mechanism, or to introduce into the host bacterium both the sequence(s) encoding the Abi and/or type II R/M mechanism(s), and the sequence(s) encoding the HsdR, HsdM or HsdS subunit(s).

In transformed bacteria produced in accordance with the invention, the sequences encoding the mechanisms of bacteriophage resistance and in particular those encoding the HsdR, HsdM or HsdS subunits in accordance with the invention, may be carried by one same DNA molecule (bacterial chromosome or plasmid) or by different DNA molecules. Several sequences, carried by the same DNA molecule, may be part of the same transcription unit, or alternatively different transcription units. They may be placed under the control of their own transcription regulatory sequences, or under the control of heterologous sequences which are active in the host bacterium.

For example, the sequences encoding the HsdR, HsdM or HsdS subunits may be expressed under the control of promoter sequences which allow constitutive expression, or, advantageously, under the control of promoter sequences which allow inducible expression, for example a cold-inducible promoter such as that described in Application FR 96/15731 in the name of INSTITUT NATIONAL DE LA RECHERCHE AGRONOMIQUE.

Natural plasmids which are preselected with the aid of nucleic acid probes in accordance with the invention, and which contain at least one sequence encoding an HsdR, HsdM or HsdS subunit of a type Ic R/M mechanism of lactic acid bacteria, may be used to carry out the process according to the invention.

A subject of the present invention is also recombinant vectors, characterized in that they result from the insertion of at least one nucleic acid sequence in accordance with the invention into a suitable vector, and in particular recombinant vectors which can be used for transforming bacteria in accordance with the invention.

According to the use envisaged, the vectors chosen may be either vectors capable of replicating and persisting in the host bacterium in the form of a plasmid, such as for example the plasmids pIL252 and pIL253 described by SIMON and CHOPIN, [Biochimie, 70, pp. 559-566, (1988)], or vectors which allow the integration of the inserts that they carry into the chromosomal DNA of the host bacterium, such as for example the vectors described in PCT Application WO 94/16086 in the name of BIOTEKNOLOGISK INSTITUT and CHR. HASSEN'S LABORATORIUM DANMARK A/S or alternatively the plasmid pG⁺ host5 described by BISWAS et al., [J. Bact., 175, pp. 3628-3635, (1995)].

More generally, plasmid vectors and integrating vectors which can be used in lactococci are described in the review of: LEENHOUTS K. J. and VENEMA G. (1993), Lactococcal plasmid vectors, in: K. G. HARDY (ed) Plasmids. A practical approach. Second Edition. IRL Press, Oxford, pp. 65-94.

If several vectors are used to introduce the different HsdR, HsdM and HsdS subunits and, optionally, other mechanisms of bacteriophage resistance into the host bacterium, mutually-compatible vectors are chosen.

The present invention may advantageously be implemented in any field in which industrial fermentation is involved, and in particular in the dairy and cheese industry. Bacterial strains having an increased resistance to bacteriophages may be produced in accordance with the invention, from strains of lactic acid bacteria of industrial value, either by selecting bacteria which naturally possess a type Ic R/M system using nucleic acid probes in accordance with the invention, or by transforming host bacteria with the aid of nucleic acid sequences in accordance with the invention.

The present invention will be better understood with the aid of the additional description which follows, and which refers to examples of cloning of sequences encoding subunits of type Ic R/M mechanisms, and of use of these sequences to increase the resistance of host bacteria to bacteriophage attacks.

### EXAMPLE 1

### Production of DNA Fragments Encoding HsdS Subunits of Type Ic R/M Mechanisms

Manipulation of the DNA, cloning and transformation of bacterial cells are, in the absence of specific details to the contrary, carried out according to the protocols described by SAMBROOK et al., [(Molecular cloning: a laboratory manual., Cold Spring Harbor Laboratory, Cold Spring Harbor N.Y. (1989)].

GAUTIER and CHOPIN [App. Environ. Microbiol. 53, 923-927 (1987)], and CHOPIN et al., [Plasmid, 11, pp. 260-263, (1984)] describe the existence of R/M type mechanisms of phage resistance, associated respectively with the plasmids pIL 103 and pIL7.

The pIL 103 DNA was digested with EcoRI, and the fragments produced were ligated to the EcoRI site of the pIL204 vector [SIMON and CHOPIN, Biochimie 70, 559-566, (1988)]. The ligation mixture was used to transform L. Lactis ssp lactis bacteria of the IL1403 strain. The bacterial colonies were selected on the basis of their resistance to the bIL67 phage. A recombinant plasmid carrying an insert of about 3.7 kb was obtained from bacterial clones which were resistant to the phage attack. This recombinant plasmid was named pIL261.

Surprisingly, sequencing the 3.7 kb insert of the pIL261 plasmid brought to light no sequence showing homology with those of the R/M mechanisms known in lactococci.

However, two reading frames, orf1 and orf2, were located in this 3.7 kb fragment.

The orf1 reading frame encodes a protein showing strong homology with the RepB proteins already identified in lactococci.

The sequence of the protein encoded by the orf2 reading frame shows no homology with known sequences of proteins of lactic acid bacteria; however, the central region and the N-terminal region of this protein contain repeated sequences which possess a certain homology with repeated consensus sequences of the HsdS subunits of type Ic R/M systems of enterobacteria and mycoplasmas.

A nucleic acid sequence containing the orf2 reading frame and the sequence of the corresponding polypeptide are represented respectively in the annexed sequence listing under the numbers SEQ ID NO: 13 and SEQ ID NO: 14.

Similarly, sequencing the pIL7 plasmid revealed a reading frame encoding a protein showing considerable homology with that encoded by the orf2 reading frame of the pIL261 plasmid, and also possessing repeated sequences resembling those of the HsdS subunits of the type I R/M systems.

A nucleic acid sequence containing this third reading frame, and the sequence of the corresponding polypeptide are represented respectively in the annexed sequence listing under the numbers SEQ ID NO: 15 and SEQ ID NO: 16.

Oligonucleotide probes, derived from the sequences encoding regions which are conserved between the sequences SEQ ID NO: 14 and SEQ ID NO: 16, were used to search for the existence of other homologous sequences of type hsdS. These probes enabled sequences of this type to be revealed on the chromosomal DNA of the IL1403 strain, and in a plasmid derived from the IL420 strain of L. lactis ssp. lactis, harboured by IL403.

The sequence of type hsdS obtained from the chromosomal DNA of the IL1403 strain, and the sequence of the corresponding polypeptide, are represented respectively in the annexed sequence listing under the numbers SEQ ID NO: 9 and SEQ ID NO: 10.

The sequence of type hsdS obtained from the plasmid derived from the IL420 strain of L. lactis ssp. lactis, and the sequence of the corresponding polypeptide, are represented respectively in the annexed sequence listing under the numbers SEQ ID NO: 11 and SEQ ID NO: 12.

### EXAMPLE 2

### Production of DNA Fragments Encoding HsdR and HsdM Subunits of Type Ic R/M Mechanisms

The regions of the chromosome of the IL1403 strain, and of the plasmid derived from the IL420 strain, in which the sequences of type hsdS were located, were entirely sequenced.

This sequencing revealed, in both cases, directly upstream of the hsdS sequence, two open reading frames encoding proteins which contain, respectively, regions showing considerable homology with conserved domains of the R and M subunits of the type Ic R/M systems of enterobacteria and of mycoplasmas. These two open reading frames have been named, respectively, because of this homology, hsdR and hsdM.

On the chromosome of the IL1403 strain, as in the plasmid derived from the IL420 strain, the open reading frame order is, from upstream to downstream: hsdR, hsdM and hsdS.

The sequence of type hsdR obtained from the chromosomal DNA of the IL1403 strain, and the sequence of the corresponding polypeptide, are represented respectively in the annexed sequence listing under the numbers SEQ ID NO: 1 and SEQ ID NO: 2.

The sequence of type hsdM obtained from the chromosomal DNA of the IL1403 strain, and the sequence of the corresponding polypeptide, are represented respectively in the annexed sequence listing under the numbers SEQ ID NO: 5 and SEQ ID NO: 6.

The sequence of type hsdR obtained from the plasmid derived from the IL420 strain of L. lactis ssp. lactis, and the sequence of the corresponding polypeptide, are represented respectively in the annexed sequence listing under the numbers SEQ ID NO: 3 and SEQ ID NO: 4.

The sequence of type hsdM obtained from the plasmid derived from the IL420 strain of L. lactis ssp. lactis, and the sequence of the corresponding polypeptide, are represented respectively in the annexed sequence listing under the numbers SEQ ID NO: 7 and SEQ ID NO: 8.

### EXAMPLE 3

### Use of Oligonucleotides Derived from the HsdR, HsdM and HsdS Sequences for Detecting Bacterial Strains which Possess Subunits of Type Ic R/M Systems

Regions of the hsdM sequences from the plasmid derived from the IL420 strain, and from the chromosome of the IL1403 strain, encoding the same peptide sequences, were identified.

The following oligonucleotides:

| | |
|---|---|
| * oligo 66 : | TTA GCA CGT ATG AAC TTA |
| * oligo 67 : | TTG GCT CGA ATG AAT TTA |

represent sequences which encode in the plasmid of the IL420 strain, and in the chromosome of the IL1403 strain, respectively, the peptide sequence (1-letter code) LARMNL.

The following oligonucleotides:

| | |
|---|---|
| * oligo 68 : | CTC TTC AAA GGT ATC CAC |
| * oligo 69 : | TTC CTC AAA GGT ATC TAC |

represent sequences complementary to the sequences encoding in the chromosome of the IL1403 strain, and in the plasmid of the IL420 strain, respectively, the peptide sequence (1-letter code) VDTFEE.

These oligonucleotides are used as PCR amplification primers (pairs 66/69 and 67/68) on 16 different strains of lactococci.

The following amplification conditions are used: the DNA extracted from each of the strains is placed in contact with deoxyribonucleotide triphosphates (each at 0.2 mM), each primer at 0.1 μM and 2.5 units of Taq polymerase (PROMEGA) in a standard buffer for Taq polymerase (PROMEGA). The reaction is carried out in a final volume of 100 μl.

After denaturing (94° C., 5 minutes), 30 cycles are carried out which alternate an annealing step at 50° C. for 30 seconds, an extension step at 72° C. for 30 seconds, and a denaturing step at 94° C. for 30 seconds.

As shown in Table I below, for 8 of the 16 strains tested (IL582, IL858, IL910, IL993, IL964, IL827, IL854 and MG1363), with one or other of the two pairs of primers, the presence of an amplification product of the expected length (about 670 bp) is observed.

TABLE I

PRIMERS

| STRAIN | 66/69 | 67/68 |
|---|---|---|
| IL827 (D) | + | + + |
| IL910 (L) | + + | − |
| IL2967 (L) | − | − |
| CNRZ194 (C) | − | − |
| CNRZ340 (D) | − | − |
| CNRZ106 (C) | − | − |
| IL2034 | − | − |
| IL854 (C) | +/− | + + |
| IL993 (C) | − | + + |
| IL858 (C) | + | + + |
| CNRZ10S (C) | − | − |
| CNRZ269 (D) | − | − |
| IL582 (L) | +/− | + + |
| IL960 (L) | − | − |
| IL964 (C) | +/− | + + |
| MG1363 (L) | + | − |

(L) ssp lactis
(D) ssp. lactis biovar. diacetylactis
(C) ssp. cremoris
+ + clearly detectable amplification product;
+ detectable amplification product;
+/− weakly detectable amplification product;
− no detectable amplification product.

The amplification products obtained from strains IL582, IL858, IL910, IL993, MG1363, IL827, IL854 and IL964 were sequenced. In strains IL582, IL858, IL993, IL827, IL854 and IL964 the sequence is very similar to the hsdM sequence from the chromosome of the IL1403 strain, and in strains IL910 and MG1363 the sequence is very similar to the hsdM sequence from the plasmid of the IL420 strain.

### EXAMPLE 4

### Resistance to Phage Attack Conferred by the HsdR, HsdM and HsdS Subunits

The strains used are: the MG1363 and IL582 strains, which each possess a sequence encoding the M subunit of a type Ic R/M system, and the IL1403 strain, which possesses on its chromosome sequences encoding the three subunits R, M and S of a type Ic R/M system.

The bacteria are cultured and infected by the phages under the conditions described by TERZAGHI and SANDINE , [Appl. Microbiol., 29, 807-815, (1975)].

The results of the infection are expressed in PFU/ml, and the efficacy of propagation (eop) is determined, for each test, by the ratio between the number of PFU/ml for the bacterial strain tested, and the number of PFU/ml for a bacterial strain chosen as control.

Table II below shows the results obtained with the c2 phage on the MG1363 strain, chosen as control, and the IL1403 strain.

TABLE II

| STRAIN | NUMBER OF PHAGES (PFU/ML) | EOP |
|---|---|---|
| MG1363 | 10⁹ | 1 |
| IL1403 | 2.8 × 10⁷ | 3 × 10⁻² |

Table III below shows the results obtained with the bIL67 phage (previously propagated on the IL1403 strain) on:

the IL1403 strain without plasmids (1), and

the IL582 strain (5) chosen as controls,

the IL1403 strain transformed with the plasmid derived from the IL420 strain, carrying hsdR, hsdM and hsdS sequences (2).

the IL1403 strain transformed with the pIL7 plasmid carrying an hsdS sequence (3)

the IL1403 strain transformed with the pIL261 plasmid carrying an hsdS sequence (4)

the IL582 strain transformed with the pIL261 plasmid carrying an hsdS sequence (6)

TABLE III

| STRAIN | NUMBER OF PHAGES (PFU/ML) | EOP |
|---|---|---|
| 1 | 6 × 10⁹ | 1 |
| 2 | 3 × 10⁴ | 5 × 10⁻⁶ |
| 3 | 3.7 × 10⁷ | 6 × 10⁻³ |
| 4 | 4 × 10³ | 6 × 10⁻⁷ |
| 5 | 4 × 10⁹ | 1 |
| 6 | 3 × 10⁴ | 1.8 × 10⁻⁵ |

These results show that:

The HsdS subunits of the different mechanisms of resistance effectively have a different specificity: the mechanism carried by the chromosome of the IL1430 strain is relatively inefficient against the bIL67 phage, whilst on the other hand, the HsdS subunits carried by the plasmids increase, to a greater or lesser degree, resistance to this phage.

The HsdS subunits encoded by the pIL7 and pIL261 plasmids may associate with the HsdR and HsdM subunits encoded by the chromosome of IL1430 to reconstitute a functional type Ic R/M complex.

1. Polypeptide which constitutes one of the HsdR, HsdM, or HsdS subunits of a mechanism of resistance to type Ic R/M bacteriophages, characterized in that said mechanism is active against the phages of lactic acid bacteria.

2. Polypeptide according to claim 1, characterized in that it is chosen from the group consisting of:
the polypeptides constituting an HsdR subunit comprising the following sequence (I):
KRLARK
the polypeptides constituting an HsdM subunit comprising at least one of the following sequences:
the sequence (II):
GLX₁FYKYLS
in which X₁ represents I or L,
the sequence (III):
ENDX₂NLNIPRYVDTFEEEE
in which X₂ represents Y or F
the polypeptides constituting an HsdS subunit comprising:
an N-terminal domain of about 150 to 180 amino acids, comprising the following sequence (IV): PX₃LRFX₄GFTX₅DDWEERKX₆
in which X₃ represents E or Q, X₄ represents E, P, D or K, X₅ represents N or D, X₆ represents L or F;
a central domain of about 50 to 80 amino acids, comprising the following sequence (V):
EQX₇KIGX₈FFKX₉LDX₁₀TIX₁₁LHQRKLDLLKEQKKGYX₁₂QKMFPKNGX₁₃KX₁₄PELRFAX₁₅FADDWEX₁₆RKLG
in which X₇ represents R or Q, X₈ represents S, N or L, X₉ represents E, H or Q, X₁₀ represents A, D or N, X₁₁ represents A or V, X₁₂ represents L or F, X₁₃ represents A or S, X₁₄ represents I or V, X₁₅ represents E or G and X₁₆ represents D or E;
a C-terminal domain of about 160 to 200 amino acids, comprising the following sequence (VI):
EQX₁₅X₁₆IGSFFKQLDX₁₇TIX₁₈LHQRKLX₁₉
in which X₁₅ represents K or Q, X₁₆ represents K or Q, X₁₇ represents N or D, X₁₈ represents T, A or V and X₁₉ represents A or D;
and/or the following sequence (VII):
GFLQKMFX₂₀
in which X₂₀ represents V or H.

3. Polypeptide according to claim 2, characterized in that it is chosen from the group consisting of:
an HsdR polypeptide corresponding to one of the sequences, respectively, represented in the annexed sequence listing under the numbers SEQ ID NO: 2 and SEQ ID NO: 4;
an HsdM polypeptide corresponding to one of the sequences, respectively, represented in the annexed sequence listing under the numbers SEQ ID NO: 6 and SEQ ID NO: 8;
an HsdS polypeptide consisting of:
an N-terminal domain of about 150 to 180 amino acids whose sequence is that of the N-terminal domain of any one of the sequences represented in the annexed sequence listing under the numbers SEQ ID NO: 10, SEQ ID NO: 12, SEQ ID NO: 14 and SEQ ID NO: 16;
a central domain of about 50 to 80 amino acids, whose sequence is that of the central domain of any one of the sequences represented in the annexed sequence listing under the numbers SEQ ID NO: 10, SEQ ID NO: 12, SEQ ID NO: 14 and SEQ ID NO: 16;
a C-terminal domain of about 160 to 200 amino acids, whose sequence is that of the C-terminal domain of any one of the sequences represented in the annexed sequence listing under the numbers SEQ ID NO: 10, SEQ ID NO: 12, SEQ ID NO: 14 and SEQ ID NO: 16.

4. Nucleic acid sequence, characterized in that it encodes a polypeptide according to any one of claims 1 to 3.

5. Nucleic acid sequence according to claim 4, characterized in that it is chosen from the group consisting of:
the hsdR sequences SEQ ID NO: 1 and SEQ ID NO: 3;
the hsdM sequences SEQ ID NO: 5 and SEQ ID NO: 7;
the hsdS sequences SEQ ID NO: 9, SEQ ID NO: 11, SEQ ID NO: 13 and SEQ ID NO: 15.

6. Nucleic acid fragment of at least 18 bp which is homologous or complementary to all or part of a nucleic acid sequence according to either of claims 4 and 5.

7. Nucleic acid fragment according to claim 6, chosen from the group consisting of the oligonucleotides which are homologous or complementary to a sequence encoding at least 6 consecutive amino acids from one of the following peptides;
TGSGKT
KRLARK
LLYGFDS
FYKYLS
LARMNL
LPHGVLFRGAAE
NLNIPRYVDTFEEEE
DDWEERK
LHQRKLDLLKEQKKGY
QKMFPKNG
PELRFA
FADDWE
IGSFFKQLD
GFLQKMF.

8. Use of at least one nucleic acid fragment according to either of claims 6 and 7, for selecting lactic acid bacteria containing a nucleic acid sequence encoding at least one subunit of a type Ic R/M mechanism.

9. Use of at least one nucleic acid fragment according to either of claims 6 and 7, for isolating and/or cloning a nucleic acid comprising a sequence according to either of claims 4 and 5.

10. Use of at least one nucleic acid sequence according to either of claims 4 and 5, to enable the expression, in a lactic acid bacterium, of at least one mechanism of resistance to type Ic R/M bacteriophages.

11. Bacterium transformed by at least one nucleic acid sequence according to either of claims 4 and 5.

12. Bacterium transformed according to claim 11, characterized in that it is, in addition, capable of expressing a mechanism of resistance to bacteriophages chosen from the Abi mechanisms and the type II R/M mechanisms.


SEQUENCE LISTING (40 sequences)

SEQ ID NO: 1 - DNA, 3050 bp, Lactococcus lactis
  CDS (44)...(3031)
aaagataacaaaaatcatgcattattgagagggagaaaaaggaatggcagaagcaaaatttgaagctgctttgatcaaaaaactagaagcagaaggatggacctatcgtaaagatttatcttatgtctctattaaggtcttagagggacactggcgtgaggtgcttaatgaaaataatgcttataaattaaatggcaaacctttgtctgatgttgaatttggcttagtgatgcaagaagttcaaaggattaaaacgccttatgatgctcaacttttattggtaggggcaggaggtgtcggctctattcctataacacgagatgacggctctaacttagaagtagagattttttatgaagatgatgtggctggtggtcggtcacgctatgaagtcgtcagccaagtgatttttgatgagctccctcatggactagcgagcaaaagaattattgatttagccttgcttattaacggaatcccagtagcgcatattgaagaaaaagatgaacacttacaaaaccaatggggcgcttttgagcaattaaaagggtatcacggagaaggcttatatgagggattatttgcttttgttcaagttcaatttatcttgagtcaacactctgccaactattttgcccgtcctaatcgtcttgaaaattataacaaaacttttgtttttggttggcgagatgagcaacagaaagatatcactgatgcgtttgtctttgcccatcaagtgttaggaatccctgcacttcatcgactagtgactgtgaacatgattccagatgcttccaatagtaatttgatggtgatgagaagttatcaaattcaagccacaagagcgattttacagcgtatgaaagaaatggaacataacgactacattcaaaaggaagggggctatatctggcataccactgggtcgggtaaaacggtgacttcttttaaagtagcccagctactagcggctgcccctaaagtaaaaaatgttttatttattgttgatcgtgtggacttagtggatcaaacgttagaaaactttaaagattttgcttacattcaatttaaaaatagaattaaaaaagtaaatggtagggagttaaaacgagagttgagccataaaggtgcttcacagatcttattaatttctgtccaaggtttaacaaaagccgttaaaaatgggctaaaaaataccgatcgaaatgtcattattatggatgaagcacatcgaagtgctaatggagaatcggttcagcttattaaaagtgcctttcaaaaaacgacttggtttgggtttacaggaactcctaatttttatagtgacgagattaatgacgttcaaaccactcgagacatttcaacccatgatatttttgggaagagactccattcttataccatcaaggatgcgattggagatggaaacgtgttagggtttgatattacttactttaatcccacgattgaaattgaatcacttgacgaagaacattccgaaaaagattatgaaaaagaagtttatcaaagtcatgtttatcgagaacaagtggtgcaagacattttaaacctttgggataaaacgtcaagtggggctttggtcgctggcaaacgagaaaagaatgtttttcaggcgatgttagctgtttcaggaaagcaagccgtggttcattactacaatctttttaaagaaaaagcccctcatttgagggtagcaatgactttttctcgagatgaatctaatcaacctggaacaaaagaacagaatgaagccttaaaaaaagcgatcaaagaatacagtagttgcttcaatgtccctagtcttttaaatgctcaagaaccggcacgggcctacatgattgatatcactaaacggctcgctcgtaaaaaaccatataaccaaggaaaagatgaggaccgattagatctagtgattgtctcggaccaactgctgacaggatttgactcaaaatatattaatacgatttatatggataaacagctgagagaagggatgttaatccaagcgatgtcccggaccaatcgaacgtttcacttgaacagtaaacctcacggaaaagttcggttctatcgtcaaggcgaacaaatgaagtcttttgtagaaaatgcccttcggatttatacacgaggggggaatgatacgcttcagggagcagatgaagactccaaaaatgaagatatccaatccttagaagacgagcatatcctggcagaaccccaaagtcatcaaatcccaaaattaacacccgctgttcaagaactaaaagcctttgcgggagaagattttagtcaaattcctcgtggtgagaaagatttaaaacaatttgtaaggctagggctagaaacacaaaatcagattcaacaattggttcaacaaggctatgaattagggaacgaaattgacctattagatgctcaaggagagtccactggtgaaaaagttcgacttgatatttctagttttgaggagtttggggctttgcaagcaaggcttaatgatgctagagaaaaattgcccgaagaggagcgtccagacctcacagaaattagagtgggcttatctttatatgaccatgagattattgactacgattggctggttgatctcttaaacctcttcatggatcagaaaactcctgaaaacaaagcttcaattgagaaacatattctcccattggatgaaatgagccaacaagagatcaaggatattatcgtagatatagaatctggggaaattaaggaacactttacgaaagcaaccttggaagatcaaagaaagcacaaacgctcggatcgacaagagttaaaaatccgtcgatgggcggccaatcaaaaagtcaatggcaatcgtattgtccaagccttcgatcttttcttaccaggtcacagcttagtagacaattctcaattatcagcgcttgtttcagaaattgaagcagaggaaaacttaagcttttttggagcctcagaatttgagactgcattaatgagcttcttcaattcactataaaaaaataaaaatactcgca

SEQ ID NO: 2 - protein, 995 aa, Lactococcus lactis
MAEAKFEAALIKKLEAEGWTYRKDLSYVSIKVLEGHWREVLNENNAYKLNGKPLSDVEFGLVMQEVQRIKTPYDAQLLLVGAGGVGSIPITRDDGSNLEVEIFYEDDVAGGRSRYEVVSQVIFDELPHGLASKRIIDLALLINGIPVAHIEEKDEHLQNQWGAFEQLKGYHGEGLYEGLFAFVQVQFILSQHSANYFARPNRLENYNKTFVFGWRDEQQKDITDAFVFAHQVLGIPALHRLVTVNMIPDASNSNLMVMRSYQIQATRAILQRMKEMEHNDYIQKEGGYIWHTTGSGKTVTSFKVAQLLAAAPKVKNVLFIVDRVDLVDQTLENFKDFAYIQFKNRIKKVNGRELKRELSHKGASQILLISVQGLTKAVKNGLKNTDRNVIIMDEAHRSANGESVQLIKSAFQKTTWFGFTGTPNFYSDEINDVQTTRDISTHDIFGKRLHSYTIKDAIGDGNVLGFDITYFNPTIEIESLDEEHSEKDYEKEVYQSHVYREQVVQDILNLWDKTSSGALVAGKREKNVFQAMLAVSGKQAVVHYYNLFKEKAPHLRVAMTFSRDESNQPGTKEQNEALKKAIKEYSSCFNVPSLLNAQEPARAYMIDITKRLARKKPYNQGKDEDRLDLVIVSDQLLTGFDSKYINTIYMDKQLREGMLIQAMSRTNRTFHLNSKPHGKVRFYRQGEQMKSFVENALRIYTRGGNDTLQGADEDSKNEDIQSLEDEHILAEPQSHQIPKLTPAVQELKAFAGEDFSQIPRGEKDLKQFVRLGLETQNQIQQLVQQGYELGNEIDLLDAQGESTGEKVRLDISSFEEFGALQARLNDAREKLPEEERPDLTEIRVGLSLYDHEIIDYDWLVDLLNLFMDQKTPENKASIEKHILPLDEMSQQEIKDIIVDIESGEIKEHFTKATLEDQRKHKRSDRQELKIRRWAANQKVNGNRIVQAFDLFLPGHSLVDNSQLSALVSEIEAEENLSFFGASEFETALMSFFNSL

SEQ ID NO: 3 - DNA, 3150 bp, Lactococcus lactis
  CDS (9)...(3116)
taaaaaatggtggcaactatggaaatgagggggatcaaatgagtcattcggaacaaatgattgaaaaccagttcatacagatcttaagtgagaaagaaaatcagtggacttatcgtccggacttgaagtcggaagaagcactttgccaaaactttagaggccatttgaaccgaataaatttagcagtattggaagagcaactattaacggacaaagaatttaagcaagtcaaagtcgagttttcacgtttgaccggaacaccttttttagcttctcaatggcttagaggagaaaacggggtggctcaagttttattagagcgagaagatggggaaaaagtgactttagaagcctttagaaataaggatatctcaggaggaacttcttcttatgaggtagttcaccaagtggtcccagattcctctagagtagatcgtggagatgtgagcttgctgattaatgggctcccaatcattcatattgagctccaaaaagagtccgctaaagacggtttcatgcaagcttattatcaaattcagcgttatgcagaagatggattttttaaagggatttacgcaaccactcaaatcatggtgattcccaataaagtcgatacccgatactttgcaagacctagtgaagataccgctgaagcctatgctcggatgaagaagtttttatttaattggcggactgaagacaatcaaacggtttccgatttgtttgattttactcgtacagttttgcggatacccgatgcccatgaattgattagccaatataccattctcgtcgatgatccaaaaaatccaaaattcctcatggctttaaggccttaccaaattcatgctattcgtaagattcgtccaaaagcggcacagcatgaaggaggattcatttggcatgcgacaggttcaggaaagaccattaccagttttgtcgcaacgaaattattagcacaaaatgcgatcggtgtcgatcgtacggtcatggttgttgatagaacagacttagatgctcaaacgcaggatgagtttacgaagtttgcctcggaataccataccggacaaacgaccggaaattcggtagccaatactttgattgttgggatcaaaaatcaaaaacagttggctcgaaacctcctatcatcaaaaaataataatacgattttagtgaccacgattcaaaaactctctgcggctatgcgaagtgcccaacaagagagtgaagaaaaaggctcgaatcaatttgagaagctgcggcaagaacatattgtttttattgttgatgaggctcatcgtgcggttagtgatgaggaaatgaaacgaattaagaaaatattacccaattctacctggtttggattaacggggacgcctatttttgaagcaaataaaaagcaagaaaatggaacctttgccagaacgacgagccagcaatacgggccactccttcactcctatacaaccaaaaatgccatggatgatggggctgtgttaggctttcaagtcgagtatcattcgctgatttcagaagaggatctagaggtgattgtcacccaactcaataaaggaaagttgccaggcgatgccctccaacaagaggaattattgcctgcggaactttatgaaaaagatgaacatattcgtaccatgttacaaaaaatctttaatcggaggagtgtggtcaaaaagttcaaggtaaagaatggttttcctacgatgtcagccattcttaccactcactcgattgctcaagccaaacatatttaccggattttaaaggaaatgaaagataatgggacactcttgaatggtcgacaatttgatgaacgtcatcgactgattgataaagatttcccaagagtagctattaccttctcaaccaatccggatcgattagaaaaaaatgaacaagacgatgaattagtagagatcatgaaagagtatgcgaaacagtttgatgcttctccttatcaagatgagaaactctacaatcaaaatattaataaacggttggcccgtaaggaaaagcaatatcaatcggatggtcagtggctagattttgttattgttgtcgatcgtttattgacaggctttgattctccagcgattctaaccctatatattgatcgagaaatgaactatcaaaagctccttcaagcgttctcaagaaccaaccgtatttatacaggaaaagattctggtttgattgtctcttttagaaaacccttcaccatgaaagagaatgtgcaaaacacgtttcgtctattctcaaatgaaaatcaaaactttgatcaactgattccaagagaatatgaagaagtcaaaaaagaatttatcgaatgttcaacactttataaacaaagcgaagctgacctatcggataatccccacgatcttaaaacgatgattgcgcaagtgagtgcttatcagaagctaggtaagagttataaagcgttccgaagctatgatcaatacgaagaggactttgaagcattttcagaagtggtggagcagttgccacaatatcgaggtaaaacggaaaatgttaaaacaaagattaaagaaatgatcgaggatgaggagcatcctgaggaggacttcgagaagcttttacaagagattgctttttcttcgcagctcaatgcgacacataaagacgtcgtggatagtttttatattaatcaacttttgaaagcgatccaattaaacgaagcaggggccgttgaaaaatttgaaaaagaaatccaacaaaaggatcctcaaattcaaaagatgtatcacaccttgaaagatcaactcgtcaatactactgaagagatcgatgtggctcaattaaaagagacttcgattcaaaatgaaattcaaagactacttcaaaaagaagctgaagaatttggattatccttcgactttttacagtctgcaatgaatgaatatcaaggcgataagaaagcgatcccttatttaacccatttactggattcgatgactcttagtaaagaagagttcgaacccaaagcgggtgaaaaatacagaagaagaccaaaagttttagaagaacgactacgacaaaactttgaacaaattcaaaaatggaaagaagaattataatggcgacaggtttaaatcaacaactatgggcttc

SEQ ID NO: 4 - protein, 1025 aa, Lactococcus lactis
MSHSEQMIENQFIQILSEKENQWTYRPDLKSEEALCQNFRGHLNRINLAVLEEQLLTDKEFKQVKVEFSRLTGTPFLASQWLRGENGVAQVLLEREDGEKVTLEAFRNKDISGGTSSYEVVHQVVPDSSRVDRGDVSLLINGLPIIHIELQKESAKDGFMQAYYQIQRYAEDGFFKGIYATTQIMVIPNKVDTRYFARPSEDTAEAYARMKKFLFNWRTEDNQTVSDLFDFTRTVLRIPDAHELISQYTILVDDPKNPKFLMALRPYQIHAIRKIRPKAAQHEGGFIWHATGSGKTITSFVATKLLAQNAIGVDRTVMVVDRTDLDAQTQDEFTKFASEYHTGQTTGNSVANTLIVGIKNQKQLARNLLSSKNNNTILVTTIQKLSAAMRSAQQESEEKGSNQFEKLRQEHIVFIVDEAHRAVSDEEMKRIKKILPNSTWFGLTGTPIFEANKKQENGTFARTTSQQYGPLLHSYTTKNAMDDGAVLGFQVEYHSLISEEDLEVIVTQLNKGKLPGDALQQEELLPAELYEKDEHIRTMLQKIFNRRSVVKKFKVKNGFPTMSAILTTHSIAQAKHIYRILKEMKDNGTLLNGRQFDERHRLIDKDFPRVAITFSTNPDRLEKNEQDDELVEIMKEYAKQFDASPYQDEKLYNQNINKRLARKEKQYQSDGQWLDFVIVVDRLLTGFDSPAILTLYIDREMNYQKLLQAFSRTNRIYTGKDSGLIVSFRKPFTMKENVQNTFRLFSNENQNFDQLIPREYEEVKKEFIECSTLYKQSEADLSDNPHDLKTMIAQVSAYQKLGKSYKAFRSYDQYEEDFEAFSEVVEQLPQYRGKTENVKTKIKEMIEDEEHPEEDFEKLLQEIAFSSQLNATHKDVVDSFYINQLLKAIQLNEAGAVEKFEKEIQQKDPQIQKMYHTLKDQLVNTTEEIDVAQLKETSIQNEIQRLLQKEAEEFGLSFDFLQSAMNEYQGDKKAIPYLTHLLDSMTLSKEEFEPKAGEKYRRRPKVLEERLRQNFEQIQKWKEEL

SEQ ID NO: 5 - DNA, 1600 bp, Lactococcus lactis
  CDS (21)...(1568)
agaaataaggagcgaataaaatggcattatcaaatgaacaaaaaaataaaatgtgggccctcttaaatcaaacccgaggacagattggtttaaccgcctataaagactacatttttggcttactattttataaatatttatcagaaaaggccacacaatggctgggagaagttttacgaggagacacttgggaaaatgtttatgatcaagatcctgtaagagccttagactatatgaaacaaaagctcggttatgccatccaaccgaaggaattttttaaagattgggaagctgccattcatgaagaacgatttaacattccaatgatttcagatacatttggccattttaatcagcaaattgcttttgaagctaaagatgattttgaaggcatctttgatgggatgcgttttgatagttcagatttaggaagtaatgcgcaagctcgagcaagtgtgatgatctcgatgattgagttactctctgcccctgagtttgatctctctacaggaggggacaccgtttcagatatctacgaatacttgttagaaaagtttgcgaccgtattggcctcagacatgggacaatactatactcccaaagaaatttcagaggtcatggctcgtattttgacttttggtaaagccgacgaagacaatttttctatttatgatcccgcggtcggttcagcttctctcttaattactaccgccagtcatatgaaacattccaatcaaaggggagcgattaaatactttggtcaagaaaaagatgccaccccttaccgattggctcgaatgaatttaatgatgcataacattgaatacaatgatattcagatccatcatgcggatactttggaaagcgattggcctgacggagtcattgaagggaaagatacgcctcgaatgtttgacgcagtgatggctaatccaccttattctgcccattggaataataaggatcgggaagatgaccctcgatttcgtgaatatggcattgcgccaaaaactaaagcggactattctttcttgttgcattgtttgtaccataccaaagagagtggccgtgtggccattattctcccacacggggtcttgtttagaggcgctgcggaaggacggattcgaaaagctttgattgataaacatcaaattgaagcggtaattggttttccagataaactctttttgaacacaggcattcccgtttgtgtcttaattcttaaaaagaatcgagcaaactctgatattctttttgtggacgccagtcaaggctttgaaaagatgaagaatcaaaaacaattgcgcccagaagatattgacaagatcactgaaacagtcattcatcgcaaggcggttgataaatatagccacttagcaacgcttgaggaagtcattgagaatgattacaacttaaatattccgcgttatgtggatacctttgaagaggaagaatcgattgacttagcggatattcaaggtcaaatagatgaagtagacgcagagattgcgaaagctaatcaaaccttagcaaactactttaaggaactgggagtgctaaaatgaaggaaagattaaaggctccagaattaaggttt

SEQ ID NO: 6 - protein, 515 aa, Lactococcus lactis
MALSNEQKNKMWALLNQTRGQIGLTAYKDYIFGLLFYKYLSEKATQWLGEVLRGDTWENVYDQDPVRALDYMKQKLGYAIQPKEFFKDWEAAIHEERFNIPMISDTFGHFNQQIAFEAKDDFEGIFDGMRFDSSDLGSNAQARASVMISMIELLSAPEFDLSTGGDTVSDIYEYLLEKFATVLASDMGQYYTPKEISEVMARILTFGKADEDNFSIYDPAVGSASLLITTASHMKHSNQRGAIKYFGQEKDATPYRLARMNLMMHNIEYNDIQIHHADTLESDWPDGVIEGKDTPRMFDAVMANPPYSAHWNNKDREDDPRFREYGIAPKTKADYSFLLHCLYHTKESGRVAIILPHGVLFRGAAEGRIRKALIDKHQIEAVIGFPDKLFLNTGIPVCVLILKKNRANSDILFVDASQGFEKMKNQKQLRPEDIDKITETVIHRKAVDKYSHLATLEEVIENDYNLNIPRYVDTFEEEESIDLADIQGQIDEVDAEIAKANQTLANYFKELGVLK

SEQ ID NO: 7 - DNA, 1700 bp, Lactococcus lactis
  CDS (48)...(1661)
aaagttttagaagaacgactacgacaaaactttgaacaaattcaaaaatggaaagaagaattataatggcgacaggtttaaatcaacaactatgggcttctgcggatatcttacgtgggaagatggatgcaagtgagtacaaaaactatttactgggattaattttctataagtacttgtctgatgcgcagctgagagaagtttatgaacaagaaaatgggaagacagataccttcccagaacgttccactcagtatgcaggcttcatggaatggtacgaagaagacaaagacgatttaatcgaaaatatccaacccaaacaaggttactttatccaacccgatcaattgttttatagttaccgcatcaaagcagacaactatgaatttaatttgaccgatttacaagcaggttttaatgagttggaacgtcaaggagaagaattcagtggattgtttgcggacattgatttaaactccacaaaattaggatcaaatgcactactacgtaatgtgacgatcactgaggttttgcgtgccttagatgagattgatttatttgaacacaatggcgatgtgattggggacgcttatgagtatttgattggagaattcgcgtcaagtgcaggtaaaaaagccggtgagttttatacgcctcaagctgtttcgaaaatcatgtcagaaattacgtcgattggacaagaaacccgtgcgccttttcatatttatgatcctgcgatggggtcaggttctttgatgcttaatatccgtcggtatctcaataatccagatcaagtgcactatcatggacaagagttaaatacgacgacctttaacttagcacgtatgaacttaattcttcatggaatagataaagaacgcatgaacctgaataacggagatactttagatgccgattggccctcagaagaaccgtatcagtttgattccgtatgcatgaaccctccttattctgcgaaatggtcagcggcagatcaatttctctctgaccctcgttttgagcgttttggaaaattagcgcctaaatctaaagcggactttgcctttctccttcacggtttttaccatttgaaagaatcgggaacaatggggattgtcctgcctcatggcgttctctttagaggagccgcggaaggaaccatccgtcaagcccttttagaaatgggagctattgatgcggtcattggcttgccggccaatatcttttttggaacgagtattccgacaacggttattattttgaagagaaaccgctctcgtcgtgatgtcttatttatcgatgcttctcaagactttgaaaaacgaaaaaatcaaaatgtcctgttggatgaacatattgataaaattgtttctatccacaaaaaacgagaagatattgaaagatatgctcatgttgcaagttttgatgagatccaagaaaatgactttaacttaaatatccctcgttatgtagatacctttgaggaagaggaaccggttgatttggttgcagtaaataccaatctccttaagatcaatgaagaattagttcaacaagagcaagtgctcttatcgatgattgacaattttgcagaaagtgaagagaatcaagccttgattgaatcgatgcgtcttcttttgagaggcggtcatgatgagtaaaaagagtccacaattaaggtttgaaggttttacggatga

SEQ ID NO: 8 - protein, 537 aa, Lactococcus lactis
MERRIIMATGLNQQLWASADILRGKMDASEYKNYLLGLIFYKYLSDAQLREVYEQENGKTDTFPERSTQYAGFMEWYEEDKDDLIENIQPKQGYFIQPDQLFYSYRIKADNYEFNLTDLQAGFNELERQGEEFSGLFADIDLNSTKLGSNALLRNVTITEVLRALDEIDLFEHNGDVIGDAYEYLIGEFASSAGKKAGEFYTPQAVSKIMSEITSIGQETRAPFHIYDPAMGSGSLMLNIRRYLNNPDQVHYHGQELNTTTFNLARMNLILHGIDKERMNLNNGDTLDADWPSEEPYQFDSVCMNPPYSAKWSAADQFLSDPRFERFGKLAPKSKADFAFLLHGFYHLKESGTMGIVLPHGVLFRGAAEGTIRQALLEMGAIDAVIGLPANIFFGTSIPTTVIILKRNRSRRDVLFIDASQDFEKRKNQNVLLDEHIDKIVSIHKKREDIERYAHVASFDEIQENDFNLNIPRYVDTFEEEEPVDLVAVNTNLLKINEELVQQEQVLLSMIDNFAESEENQALIESMRLLLRGGHDE

SEQ ID NO: 9 - DNA, 1400 bp, Lactococcus lactis
  CDS (15)...(1385)
ctgggagtgctaaaatgaaggaaagattaaaggctccagaattaaggtttgatggttttacggatgattgggaagagcgtaagttattagacaatgtagaaaaagtattacattatcgtggtaaaagccccgctaagtttggaatggaatggggtacagaaggttatcttgttctttctgctttgaatgttaaaaatggatacattgataaatctgttgaagcaaagtatggggatcatgaattatttgacagatggatggggaacaatcggttagaaaaaggagatgttgtttttacaacagaagctcctttaggcaatgtagctcaagtacccgataataacggatatatattaaaccaaagagcggttgcatttaaatctttgcaagaaacagatgataatttctttgcgcaattactacgaagtcctattgttcaaaatacattaaaagctagttcttctgggggaactgccaaaggtattggaatgaaagaatttgctaaacttaatgctcgagttcccgaaacacatgaagaacaacgaaaaatagggttattcttcaaacagctagacgacactatcgttcttcatcaacgtaagttagatcttctcaaagagcagaaaaaaggatacttgcaaaaaatgttccccaaaaatggttcaaaaattcctgaattgagatttgcggagtttgctgacgattgggaagaacgtaagttgggtgaagttgcaacatttctaaacggacgagcatataagcaagatgaattactggattcaggtaaatataaagtgcttcgcgtcggcaatttttataccaatgattcttggtactactctaatatggagcttggtgataaatattatgttgataaaggtgatctagtttatacttggtcagcgacatttggcccacatatttggagtggcgaaaaagttatttaccattatcacatctggaaagtcgagctttctaaatttcttgataggaattttacattgcaacttttagaagcggataaagcaagattgttatccagtacaaatggttcaacaatgattcatgtaaccaagggagatatggaaagtaaaattgtttctattcctaatattgatgagcaaaaacaaattggttcattcttcaaacaactcgacaacactatcacccttcatcaacgtaagttagatttgttgaaagagcagaaaaaaggatttctacaaaaaatgtttcatttaacaaatttaggagccttcactatggaaactatacaaaaatatttaaagataattgttgaaagtaattatcttcatcaacgtaagttagacttgcttaaggagcaaaaaagaattattacaaataatgtttattcattttttgtataatataaagcgatttaa

SEQ ID NO: 10 - protein, 456 aa, Lactococcus lactis
MKERLKAPELRFDGFTDDWEERKLLDNVEKVLHYRGKSPAKFGMEWGTEGYLVLSALNVKNGYIDKSVEAKYGDHELFDRWMGNNRLEKGDVVFTTEAPLGNVAQVPDNNGYILNQRAVAFKSLQETDDNFFAQLLRSPIVQNTLKASSSGGTAKGIGMKEFAKLNARVPETHEEQRKIGLFFKQLDDTIVLHQRKLDLLKEQKKGYLQKMFPKNGSKIPELRFAEFADDWEERKLGEVATFLNGRAYKQDELLDSGKYKVLRVGNFYTNDSWYYSNMELGDKYYVDKGDLVYTWSATFGPHIWSGEKVIYHYHIWKVELSKFLDRNFTLQLLEADKARLLSSTNGSTMIHVTKGDMESKIVSIPNIDEQKQIGSFFKQLDNTITLHQRKLDLLKEQKKGFLQKMFHLTNLGAFTMETIQKYLKIIVESNYLHQRKLDLLKEQKRIITNNVYSFFV

SEQ ID NO: 11 - DNA, 1308 bp, Lactococcus lactis
  CDS (44)...(1258)
gccttgattgaatcgatgcgtcttcttttgagaggcggtcatgatgagtaaaaagagtccacaattaaggtttgaaggttttacggatgattgggaagaacgtaagtttggagaagtttggaaaaaatcaagtgagcgcaatttaaatttagaatattcaccgaagcaagttttatcagtggctcaaatgaaattaaatccatctaatagaaatgaacaggatgattatatgaaaacatataatgtgcttcataaaggtgatattgcatttgaaggtaacaagtcaaagagttttgcatttggtaggtttgttctagatgatttacaggatgggatagtctcgcatgtattttatgtatatcgccctatttgtaaaatggatacagattttatgatagtttatataaataatgaatctgtaatgaagtatcttttagttaaggcaactactaaaactttgatgatgactactttaaatactaaagatattgttaaaccaaaactaaacttacctagtcttgaagaacaacaaaagatcggttcattcttcaaacagttagatgccactatcgctcttcatcaacgtaagctagatttgttgaaagaacagaaaaaaggctacttccaaaaaatgttccctaaaaatggtgccaaagttcctgaattgcgatttgcggggtttgctgacgattgggaagatcgtaagttaggtgaattagctagtttttcaaaaggcaatggatatacaaaaaatgatttagtcgaatttggagatccaataattttatatggtcgcttatatacaaaatatgaaacagttattgagaaagtagatacatttgtaaataagaaagataaatcaataattagtggaggatctgaagtcatagtgcctgcatcaggagaatcttcagaagatatttctagagcttcagttgttgggaaatcaggtataattttaggcggagatttaaatataattaaaccagtaaattatattgattctatttttttagctttaactatttctaatggatctcaacaaaaagaaatgtctaagagagctcaaggaaaatctgtagttcatctgcataactctgatctaaaacaagtaaatattttatatccaaaattaggagaacaacaaaaaatcggttcattcttcaaacaactagataacactatcgttcttcatcaacgtaagttagattttttgaaagagcagaaaaaaggctttttacaaaagatgtttgtttagggtctataattagataataacccctcacaaatcccattaaaatagcccct

SEQ ID NO: 12 - protein, 404 aa, Lactococcus lactis
MSKKSPQLRFEGFTDDWEERKFGEVWKKSSERNLNLEYSPKQVLSVAQMKLNPSNRNEQDDYMKTYNVLHKGDIAFEGNKSKSFAFGRFVLDDLQDGIVSHVFYVYRPICKMDTDFMIVYINNESVMKYLLVKATTKTLMMTTLNTKDIVKPKLNLPSLEEQQKIGSFFKQLDATIALHQRKLDLLKEQKKGYFQKMFPKNGAKVPELRFAGFADDWEDRKLGELASFSKGNGYTKNDLVEFGDPIILYGRLYTKYETVIEKVDTFVNKKDKSIISGGSEVIVPASGESSEDISRASVVGKSGIILGGDLNIIKPVNYIDSIFLALTISNGSQQKEMSKRAQGKSVVHLHNSDLKQVNILYPKLGEQQKIGSFFKQLDNTIVLHQRKLDFLKEQKKGFLQKMFV

SEQ ID NO: 13 - DNA, 1300 bp, Lactococcus lactis
  CDS (11)...(1288)
ggtggggactatggcgaaaatagatgattcagttaaaaagagagttccagaattaaggtttccgggatttacgaatgattgggaagagcgtaagttttttgaaagtatagcttcaacaatagattttagaggtagaactcctaaaaagttaggcatggactggagtgattctggatatttagctttatccgctttgaacgtaaaaaatggatatattgatccgttagctgatgctcactatggtgatgagaaattatacagaaaatggatgtcaggaagagaactaaaaaaaggacaggttctctttacaacagaggctcctatgggaaatgtcgctcaagtacctgatgataacggatatattttaagtcaaagaactgtagcatttgagaccaaagaagatatgatgactaatgacttcttagctgtattattaaaatctcctttagttttcaataatttatcagcattatcaagcggtggaactgctaaaggggttagtcaaaaatcattaaaaggactatctataactgtcccattggatattgacgagcaacaaaaaatcggttcattcttcaaacatttagatgacactatcgctcttcatcaacgtaagttggatttactcaaggaacagaaaaaaggctacttgcaaaaaatgttccctaaaaatggtgccaaagttcctgaattgcgatttgcggggtttgctgacgattgggaagagcgtaagttgggtgatatagctcccttacgtggcggttatgcattcaaaagctctaaattcaggaaaactggtgtgccaattgttagaatttcaaatattctttccagcggggaggtaggtggagattttgcatattatgatgaacaggataaggatgacaaatatattcttccagataaatcagcagtcctagccatgtcaggtgcaacaacaggtaaggtatctatactatctcaaactgattatgacaaggtctatcaaaaccagcgagttggttattttcagtctgtagactatattgactacgggtttatttccacaatcgtccgttcagaattattcatgatgcaacttgagtctgttctagtttcaggcgctcagccaaatgtatcgtcaaaagaaattgattcgtttaattttatgattcccatattagttcaggagcaacaaaaaatcggttcattcttcaaacagttagatgacactatcgctcttcatcaacgtaagctagatttgttgaaagaacagaaaaaaggctttttacaaaagatgtttgtttagggtctataatta

SEQ ID NO: 14 - protein, 425 aa, Lactococcus lactis
MAKIDDSVKKRVPELRFPGFTNDWEERKFFESIASTIDFRGRTPKKLGMDWSDSGYLALSALNVKNGYIDPLADAHYGDEKLYRKWMSGRELKKGQVLFTTEAPMGNVAQVPDDNGYILSQRTVAFETKEDMMTNDFLAVLLKSPLVFNNLSALSSGGTAKGVSQKSLKGLSITVPLDIDEQQKIGSFFKHLDDTIALHQRKLDLLKEQKKGYLQKMFPKNGAKVPELRFAGFADDWEERKLGDIAPLRGGYAFKSSKFRKTGVPIVRISNILSSGEVGGDFAYYDEQDKDDKYILPDKSAVLAMSGATTGKVSILSQTDYDKVYQNQRVGYFQSVDYIDYGFISTIVRSELFMMQLESVLVSGAQPNVSSKEIDSFNFMIPILVQEQQKIGSFFKQLDDTIALHQRKLDLLKEQKKGFLQKMFV

SEQ ID NO: 15 - DNA, 1250 bp, Lactococcus lactis
  CDS (46)...(1233)
aaaaaggatagaagaacaagaaaaaccaagaaagtggtggggactatggcgaaaatagatgattcagttaaaaagaaagttcctgaattgcgatttaaaggatttacgaatgattgggaagagcgtaagttaggagaattatctaatattgttggtggtggaacaccaagtacatcgaaccctgaatactgggacggtgatattgattggtatgcaccagctgaaattggagaacaaagttatgttagcaaaagtaaaaagactattactgaactaggtctaaagaagagttcagctcgaattttaccagtaggaaccgtcttatttacttctcgtgctggtatcggaaacaccgctatattagccaaagaagctacaactaatcaagggtttcaatcaattgttcctgatcaaaataaacttgatagttattttattttttcaagaactaatgagctcaagcgatatggggaagtcactggtgcaggatctacttttgttgaggtttcaggtaagcaaatgtcgaaaatgtcgattatggtacctgaactttcagaacaacaaaaaattggtaacttctttaaagagttagacaacactatcgctcttcatcagcgtaagttagatttgttgaaagaacagaaaaaaggctacttgcaaaaaatgttccctaaaaatggtgccaaagttcctgaattgcgatttgcggggtttgctgacgattgggaagagcgtaagttaggagatattactaaaataagcaccggaaaattagatgcaaatgcaatggttgaaaatggcaagtatgatttttatacttctggaattaaaaaatataggatcgatgtagcagcgtttgaaggaccgtccattacaattgcaggtaatggagcaacagttggatacatgcatttagcggataataagttcaatgcctatcaacgaacttatgtgctccaagagtttctagtagatagaagttttatattttcagaaattggaaataagctgcctaaaaaaataaaacaggaagctcgtacaggaaatattccgtacattgttatggacatgttgactgaactgaagctatcgattcctcagaataattcagagcaacaaaaaataggttcattctttaaacagctagatgacactatcgctcttcatcaacgtaagttagcatttgttgaaagaacagaaaacacaggctttttacaaaagatgtttgtttagggtctataattagataa

SEQ ID NO: 16 - protein, 395 aa, Lactococcus lactis
MAKIDDSVKKKVPELRFKGFTNDWEERKLGELSNIVGGGTPSTSNPEYWDGDIDWYAPAEIGEQSYVSKSKKTITELGLKKSSARILPVGTVLFTSRAGIGNTAILAKEATTNQGFQSIVPDQNKLDSYFIFSRTNELKRYGEVTGAGSTFVEVSGKQMSKMSIMVPELSEQQKIGNFFKELDNTIALHQRKLDLLKEQKKGYLQKMFPKNGAKVPELRFAGFADDWEERKLGDITKISTGKLDANAMVENGKYDFYTSGIKKYRIDVAAFEGPSITIAGNGATVGYMHLADNKFNAYQRTYVLQEFLVDRSFIFSEIGNKLPKKIKQEARTGNIPYIVMDMLTELKLSIPQNNSEQQKIGSFFKQLDDTIALHQRKLAFVERTENTGFLQKMFV

SEQ ID NO: 17 - protein, 6 aa, Artificial Sequence
  HsdR subunit
KRLARK

SEQ ID NO: 18 - protein, 9 aa, Artificial Sequence
  HsdM subunit;
GLXFYKYLS

SEQ ID NO: 19 - protein, 19 aa, Artificial Sequence
  UNSURE (4)...(4): Xaa= Tyr or Phe
ENDXNLNIPRYVDTFEEEE

SEQ ID NO: 20 - protein, 18 aa, Artificial Sequence
  An N-terminal domain
PZLRFXGFTBDDWEERKX

SEQ ID NO: 21 - protein, 63 aa, Artificial Sequence
  A central domain
EQXKIGXFFKXLDXTIXLHQRKLDLLKEQKKGYXQKMFPKNGXKXPELRFAXFADDWEXRKLG

SEQ ID NO: 22 - protein, 24 aa, Artificial Sequence
  a C-terminal domain
EQXXIGSFFKQLDBTIXLHQRKLX

SEQ ID NO: 23 - protein, 8 aa, Artificial Sequence
  a C-terminal Domain
GFLQKMFX

SEQ ID NO: 24 - protein, 6 aa, Artificial Sequence
  HsdR subunit
TGSGKT

SEQ ID NO: 25 - protein, 7 aa, Artificial Sequence
  HsdR subunit
LLTGFDS

SEQ ID NO: 26 - protein, 6 aa, Artificial Sequence
  HsdM subunit
FYKYLS

SEQ ID NO: 27 - protein, 6 aa, Artificial Sequence
  HsdM subunit
LARMNL

SEQ ID NO: 28 - protein, 12 aa, Artificial Sequence
  HsdM subunit
LPHGVLFRGAAE

SEQ ID NO: 29 - protein, 15 aa, Artificial Sequence
  HsdM subunit
NLNIPRYVDTFEEEE

SEQ ID NO: 30 - protein, 7 aa, Artificial Sequence
  HsdS subunit
DDWEERK

SEQ ID NO: 31 - protein, 16 aa, Artificial Sequence
  HsdS subunit
LHQRKLDLLKEQKKGY

SEQ ID NO: 32 - protein, 8 aa, Artificial Sequence
  HsdS subunit
QKMFPKNG

SEQ ID NO: 33 - protein, 6 aa, Artificial Sequence
  HsdS subunit
PELRFA

SEQ ID NO: 34 - protein, 6 aa, Artificial Sequence
  HsdS subunit
FADDWE

SEQ ID NO: 35 - protein, 9 aa, Artificial Sequence
  HsdS subunit
IGSFFKQLD

SEQ ID NO: 36 - protein, 7 aa, Artificial Sequence
  HsdS subunit
GFLQKMF

SEQ ID NO: 37 - DNA, 18 bp, Artificial Sequence
  Oligonucleotide primer
ttagcacgtatgaactta

SEQ ID NO: 38 - DNA, 18 bp, Artificial Sequence
  Oligonucleotide primer
ttggctcgaatgaattta

SEQ ID NO: 39 - DNA, 18 bp, Artificial Sequence
  Oligonucleotide primer
ctcttcaaaggtatccac

SEQ ID NO: 40 - DNA, 18 bp, Artificial Sequence
  Oligonucleotide primer
ttcctcaaaggtatctac